(19) United States
(12) Patent Application Publication
(10) Pub. No.: US 2020/0258627 A1
(43) Pub. Date: Aug. 13, 2020

(54) SYSTEMS, DEVICES, SOFTWARE, AND METHODS FOR A PLATFORM ARCHITECTURE

(71) Applicant: Genetesis, Inc., Mason, OH (US)

(72) Inventors: Emmanuel T. SETEGN, Mason, OH (US); Peeyush SHRIVASTAVA, Mason, OH (US); Rhea MALHOTRA, Mason, OH (US); Vineet Naveen ERASALA, Mason, OH (US); Raj MUCHHALA, Mason, OH (US); Benjamin Donaldson MOORE, Mason, OH (US)

(21) Appl. No.: 16/271,705

(22) Filed: Feb. 8, 2019

(51) Int. Cl. G16H 50/20; A61B 5/04; G06N 20/00; G16H 10/60

(52) U.S. Cl. CPC G16H 50/20; G16H 10/60; G06N 20/00; A61B 5/04007

(57) ABSTRACT

Described herein are methods, software, systems and devices that include a set of hardware and software tools employed to rapidly rule-out patients that present to, for example, the emergency room and observation clinical decision units with chest pain, for coronary artery disease.

### BACKGROUND

Many medical centers and individual healthcare providers utilize computer based systems to manage patient data.

### SUMMARY

Described herein are systems, devices, software, and methods for providing a healthcare platform. In some embodiments, the described devices, software, and methods employ electromagnetic field (EMF) sensing and analysis hardware and software tools that capture and analyze a patient sensed EMF.

Described herein is a healthcare platform comprising: an electromagnetic field sensing system configured to sense an electromagnetic field data associated with an individual; a healthcare provider portal and a patient portal, the healthcare provider portal configured to be used by a healthcare provider of the individual and the patient portal configured to be used by the individual; and a server configured to operatively communicate with the healthcare provider portal, the patient portal, or both, the server encoded with software modules including: a data ingestion module configured to receive the electromagnetic field data; a service module configured to provide at least one healthcare service that is accessed through either the healthcare provider portal, the patient portal, or both, the healthcare service related to the electromagnetic field data that is sensed; an interface module that provides the healthcare provider portal, the patient portal, or both with access to the healthcare service, the interface module comprising an application programming interface. In some embodiments, the electromagnetic field sensing system comprises an array of sensors configured to detect electromagnetic fields, including optically pumped magnetometer sensors, magnetic induction sensors, magneto-resistive sensors, SQUID sensors, or any combination of these. In some embodiments, the electromagnetic field sensing system comprises an ambient electromagnetic shield. In some embodiments, the electromagnetic shield comprises a bore through which the body of the individual is passed. In some embodiments, the server is further encoded with an analysis module that utilizes machine learning to analyze the electromagnetic field data thereby generating an analysis result, and wherein the analysis module determines a diagnosis of the individual based on the analysis result. In some embodiments, the server is further encoded with a graphic module configured to generate a graphic representation of the electromagnetic field data that is sensed. In some embodiments, the at least one healthcare service comprises a graphic representation of a sensed electromagnetic field. In some embodiments, the at least one healthcare service comprises an interactive electronic medical record. In some embodiments, the at least one healthcare service comprises an interactive medical image. In some embodiments, the at least one healthcare service comprises raw sensed electromagnetic field data. In some embodiments, the at least one healthcare service comprises a global reader service which provides an interpretation of a medical image. In some embodiments, the at least one healthcare service comprises an interactive electronic medical record management service. In some embodiments, the at least one healthcare service comprises an analytic module configured to analyze the electromagnetic field data and generate an analysis result. In some embodiments, the at least one healthcare services comprises a diagnostic module that identifies a diagnosis based on the analysis result. In some embodiments, the at least one healthcare service comprises a mapping module configured to generate an electric current map based on the electromagnetic field data. In some embodiments, the healthcare provider portal provides a communication interface configured to provide at least one of text, audio, and video transmissions from the healthcare provider portal to the patient portal. In some embodiments, the patient portal provides a communication interface configured to provide at least one of text, audio, and video transmissions from the patient portal to another patient portal. In some embodiments, the application programming interface provides a portal for encoding protocols for the behavior of the interface. In some embodiments, the protocols are configured to cause the software modules to integrate with a customized healthcare provider portal. In some embodiments, the protocols are configured to cause the plurality of software modules to integrate with a customized patient portal. In some embodiments, the protocols are configured to generate a user authentication system. In some embodiments, the electromagnetic field sensing device is configured to sense an electromagnetic field associated with a heart of a patient.

Described herein is a computer implemented method comprising: sensing electromagnetic field data associated with an individual; receiving the electromagnetic field data with an ingestion module of a server encoded with a service module that provides at least one healthcare service related to the electromagnetic field that is sensed; providing access to the service module to a healthcare provider portal and a patient portal through an interface module, wherein the interface module comprises an application programming interface. In some embodiments, the electromagnetic field sensing system comprises an array of sensors configured to detect electromagnetic fields, including optically pumped magnetometer sensors, magnetic induction sensors, magneto-resistive sensors, SQUID sensors, or any combination of these. In some embodiments, the electromagnetic field sensing system comprises an ambient electromagnetic shield. In some embodiments, the electromagnetic shield comprises a bore through which the body of the individual is passed. In some embodiments, the server is further encoded with an analysis module that utilizes machine learning to analyze the electromagnetic field data thereby generating an analysis result, and wherein the analysis module determines a diagnosis of the individual based on the analysis result. In some embodiments, the server is further encoded with a graphic module configured to generate a graphic representation of the electromagnetic field data that is sensed. In some embodiments, the at least one healthcare service comprises a graphic representation of a sensed electromagnetic field. In some embodiments, the at least one healthcare service comprises an interactive electronic medical record. In some embodiments, the at least one healthcare service comprises an interactive medical image. In some embodiments, the at least one healthcare service comprises raw sensed electromagnetic field data. In some embodiments, the at least one healthcare service comprises a global reader service which provides an interpretation of a medical image. In some embodiments, the at least one healthcare service comprises an interactive electronic medical record management service. In some embodiments, the at least one healthcare service comprises an analytic module configured to analyze the electromagnetic field data and generate an analysis result. In some embodiments, the at least one healthcare services comprises a diagnostic module that identifies a diagnosis based on the analysis result. In some embodiments, the at least one healthcare service comprises a mapping module configured to generate an electric current map based on the electromagnetic field data. In some embodiments, the healthcare provider portal provides a communication interface configured to provide at least one of text, audio, and video transmissions from the healthcare provider portal to the patient portal. In some embodiments, the patient portal provides a communication interface configured to provide at least one of text, audio, and video transmissions from the patient portal to another patient portal. In some embodiments, the application programming interface provides a portal for encoding protocols for the behavior of the interface. In some embodiments, the protocols are configured to cause the software modules to integrate with a customized healthcare provider portal. In some embodiments, the protocols are configured to cause the plurality of software modules to integrate with a customized patient portal. In some embodiments, the protocols are configured to generate a user authentication system. In some embodiments, the electromagnetic field sensing device is configured to sense an electromagnetic field associated with a heart of a patient.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 depicts an example environment that can be employed to execute implementations of the present disclosure.

FIG. 2 depicts an example platform architecture that can be employed according to implementations of the present disclosure.

FIG. 3 depicts a schematic representation of an exemplary medical device that can be employed according to implementations of the present disclosure.

FIG. 4 depicts an exemplary embodiment of a medical device that can be employed according to implementations of the present disclosure.

FIGS. 5A and 5B depict schematic examples of neural network architecture in terms of flow of data within the neural network.

FIG. 6 depicts a schematic representing an exemplary machine learning software module.

FIG. 7 depicts a computer control system that is programmed or otherwise configured to implement methods according to implementations of the present disclosure.

### DETAILED DESCRIPTION

Described herein is a platform that includes a set of hardware and software tools employed to capture, analyze, and report results from collected patient magnetic fields. In some embodiments, a platform as described herein includes an EMF sensing system which further includes one or more hardware (device(s)) and software. In some embodiments, a platform as described herein comprises at least one health care provider portal and a server configured to provide at least one healthcare related service.

In some embodiments, the described platform is employed to provide results quickly, (e.g., within one hour) after an EMF scan is taken. Results may include suggesting further testing or a definitive ruling out of a patient. In some embodiments, the described platform is employed to reduce hospital burden with low to intermediate risk patients as well as streamlining certain administrative or healthcare finance tasks such as, for example, billing or insurance form submission.

In some embodiments, the described platform is deployed as a service (PaaS) and cognitive engine employed to unify a set of disjointed services in, for example, a hospital to streamline medical device usage process. In some embodiments, the described platform performs functions, such as ordering, scanning, image and signal processing, reader image analysis, and reporting. These functions can be broadly extended to many medical devices deployed in a hospital setting to collect a wide array of unique signals, e.g., ECG, magnetocardiography, magnetoencephalography, magnetic resonance imaging (MM), computerized tomography (CT), and so forth. In some embodiments, devices are preconfigured to interact with RESTful API services provided through the employed PaaS. In some embodiments, devices are connected to an existing Electronic Health Record (EHR) system to associate scans taken with a respective patient. For example, in some embodiments, when a scan is completed, a device uploads the data to the employed PaaS for processing and storage. In some embodiments, the data is analyzed by a healthcare provider who has access to the set of signals, images and tools used to analyze different types of signals or images. In some embodiments, once decided on scan quality, diagnosis, and noting any other additional comments, the healthcare provider may submit a report that is then accessible by, for example, an ordering healthcare provider, with patient demographics, scan information, signal and image metrics and parameters, and a machine-learning based score.

In various embodiments, the platforms, systems, media, and methods described herein include a cloud computing environment. In some embodiments, a cloud computing environment comprises one or more computing processors.

While various embodiments are shown and described herein, it will be obvious to those skilled in the art that such embodiments are provided by way of example only. It should be understood that various alternatives to the embodiments herein in some embodiments are employed.

### Certain Definitions

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

As used herein, the phrases “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

As used herein, the term “about” may mean the referenced numeric indication plus or minus 15% of that referenced numeric indication.

In general, the term “software” as used herein comprises computer readable and executable instructions that may be executed by a computer processor. In some embodiments a “software module” comprises computer readable and executable instructions and may, in some embodiments described herein make up a portion of software or may in some embodiments be a stand-alone item. In various embodiments, software and/or a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

A “managed physician” includes a user on the described platform that is to read and interpret results received from, for example, an EMF sensing device or system.

A “magnetocardiogram” or “MCG” is a visual representation of the magnetic fields produced by the electrical activity of the heart. An MCG as used herein includes an MCG generated from any technique that determines one or more magnetic fields associated with a heart of an individual including techniques as described herein using one or more EMF sensors as well as traditional magnetic resonance imaging techniques. A “CardioFlux” is a brand name of a system such as the systems described herein that is configured to sense an EMF associated with a patient and in some embodiments uses the sensed EMF to generate an MCG or other visual representation of an EMF. A CardioFlux system, in some embodiments, includes or is operatively coupled to software configured to analyze a sensed EMF and in some embodiments is configured to determine a diagnosis of a patient based on a sensed EMF from the patient.

“Amazon Web Services” or “AWS” is an on demand cloud computing platform.

A “global reader portal” or “GRP” is a user portal in a platform as described herein and in some embodiments provides a managed physician with the ability to view medical data including, for example, one or more medical images and provide one or more interpretations of the one or more medical images.

A “site reader portal” or “SRP” is a user portal in a platform as described herein and in some embodiments provides authorized site users with the ability to view medical data including, for example, raw medical data, interpretation results, and/or patient demographic information.

An “application programming interface” or “API” includes a set of subroutine definitions, communication protocols and tools for building software. In some embodiments, an API provides an authorized user the ability to integrate software into a platform as described herein in order to, for example, customize one or more features of the platform.

“Microservices” are a software architecture style in which complex applications are composed of small independent processes communicating with each other, using language agnostic APIs.

An “API Gateway” is an exposed set of one or more API endpoints that coordinate a set of calls to different microservices.

“Representational State Transfer” or “REST” is an architectural style that defines a set of constraints to be used for creating web services and provides interoperability between computer systems and the Internet.

“JSON Web Token” or “JWT Token” is a JSON-based open standard (RFC 7519) for creating access tokens that assert some number of claims and may include user information including encrypted user information.

“Electromagnetic field” or “EMF” data includes EMF measurements and simulations of EMF measurements.

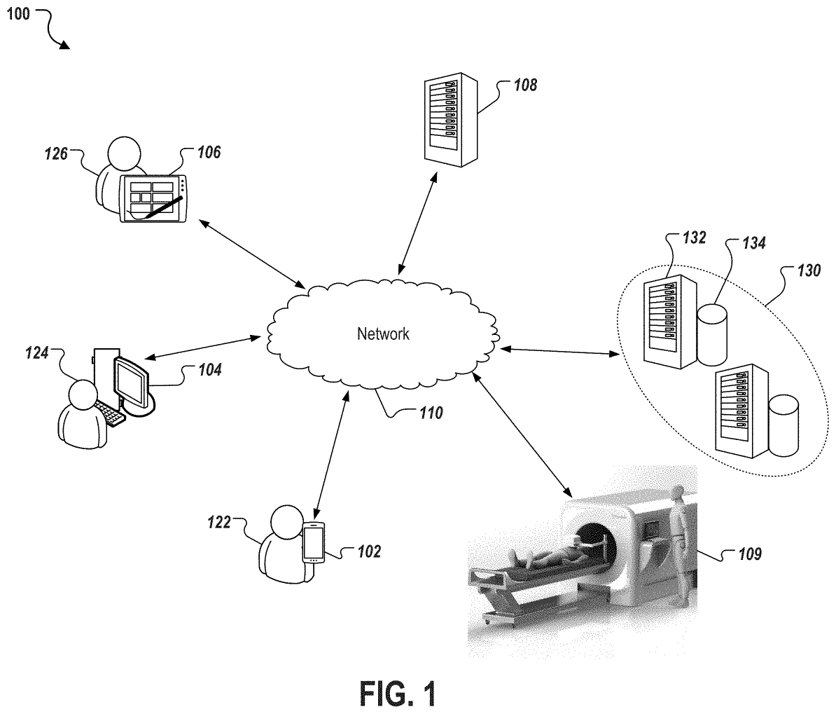

FIG. 1 depicts an example environment that can be employed to execute implementations of one or more embodiments of the platform 100 of the present disclosure. The example platform 100 includes computing devices 102, 104, 106, 108, medical device or system 109, a back-end system 130, and a network 110. In some embodiments, the network 110 includes a local area network (LAN), wide area network (WAN), the Internet, or a combination thereof, and connects web sites, devices (e.g., the computing devices 102, 104, 106, 108 and the medical device or system 109) and back-end systems (e.g., the back-end system 130). In some embodiments, the network 110 can be accessed over a wired and/or a wireless communications link. For example, mobile computing devices (e.g., the smartphone device 102 and the tablet device 106), can use a cellular network to access the network 110. In some embodiments, the users 122-126 includes physicians, patients, network technicians including network administrators and authorized programmers, nurses, residents, hospital administrators, insurers, and any other healthcare provider.

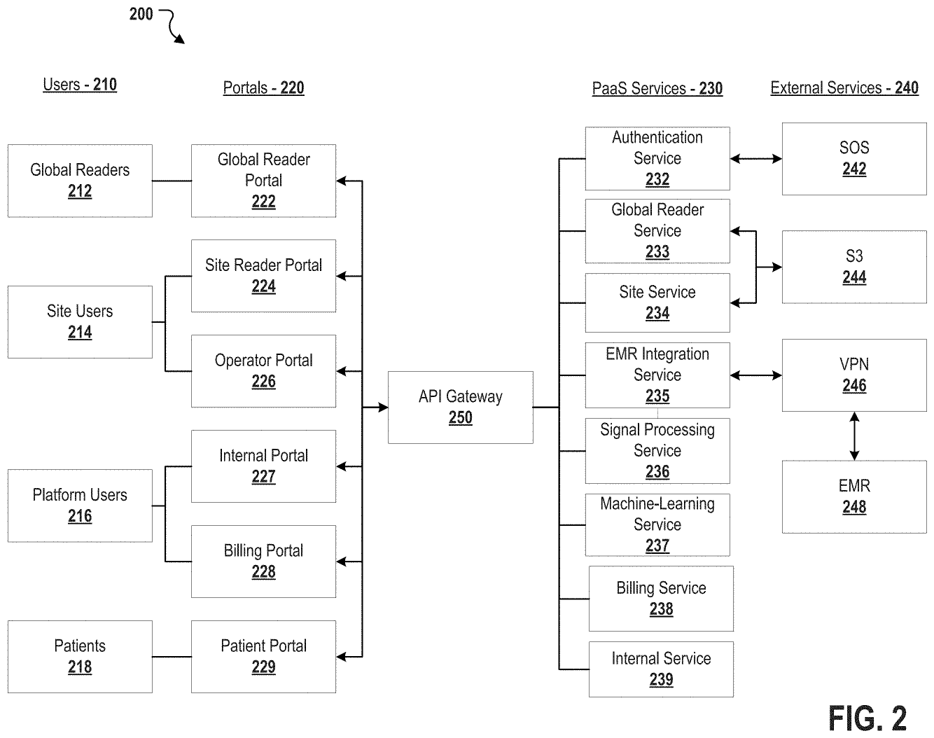

In the depicted example, the back-end system 130 includes at least one server system 132 and a data store 134. In some embodiments, the at least one server system 132 hosts one or more computer-implemented services and portals employed within the described platform, such as described in FIG. 2, that users 122-126 can interact with using the respective computing devices 102-106. For example, the computing devices 102-106 may be used by respective users 122-126 to generate and retrieve reports regarding patient scans taken by the medical device or system 109 through services hosted by the back-end system 130 (see FIG. 2). In some embodiments, the back-end system 130 provides an API service with which the server computing device 108 may communicate.

In some embodiments, back-end system 130 includes server-class hardware type devices. In some embodiments, back-end system 130 includes computer systems using clustered computers and components to act as a single pool of seamless resources when accessed through the network 110. For example, such embodiments may be used in data center, cloud computing, storage area network (SAN), and network attached storage (NAS) applications. In some embodiments, back-end system 130 is deployed using a virtual machine(s).

In some embodiments, the computing devices 102, 104, 106 include any appropriate type of computing device, such as a desktop computer, a laptop computer, a handheld computer, a tablet computer, a personal digital assistant (PDA), a cellular telephone, a network appliance, a camera, a smart phone, an enhanced general packet radio service (EGPRS) mobile phone, a media player, a navigation device, an email device, a game console, or an appropriate combination of any two or more of these devices or other data processing devices. In the depicted example, the computing device 102 is a smartphone, the computing device 104 is a desktop computing device, and the computing device 106 is a tablet-computing device. In some embodiments, the server computing device 108 includes any appropriate type of computing device, such as described above for computing devices 102-106 as well as computing devices with server-class hardware. In some embodiments, the server computing device 108 includes computer systems using clustered computers and components to act as a single pool of seamless resources. It is contemplated, however, that implementations of the present disclosure can be realized with any of the appropriate computing devices, such as those mentioned previously.

In some embodiments, the medical device or system 109 comprises an array, such as a sensor array and a shield. In some embodiments, the medical device or system 109 comprises a base unit and an array, such as a sensor array. In some embodiments, the medical device or system 109 senses an electromagnetic field associated with one or more tissues or one or more organs of an individual. In some embodiments of the devices 109, sensed electromagnetic field data associated with a heart is used to generate a magnetocardiogram. In these embodiments, the devices 109 comprise a magnetocardiograph which may, for example, be a passive, noninvasive bioelectric measurement tool intended to detect, record, and display magnetic fields that are naturally generated by electrical activity of a heart. It should be understood that in some embodiments, an EMF that is sensed is associated with a brain of an individual and/or component of a nervous system of an individual (including both central and peripheral nervous systems). In some embodiments, an EMF that is sensed is associated with an organ of an individual, and/or a tissue of an individual, and/or a portion of a body of an individual, and/or an entire body of an individual.

In some embodiments, the medical device or system 109 comprises at least one sensor, such as an optically pumped magnetometer (OPM) as a measurement tool, which may use nonradioactive self-contained alkali metal cells coupled with a closed pumping laser and photodetector setup to measure minute magnetic fields. In some embodiments, medical device or system 109 comprises an array of two or more sensors. In some embodiments comprising an array, the two or more sensors of the array are the same type of EMF sensor, and, in some embodiments, an array of sensors comprises at least two different sensors. Non-limiting examples of EMF sensors suitable for use with the exemplary medical device or system 109 include optically pumped magnetometer sensors, magnetic induction sensors, magneto-resistive sensors, and SQUID sensors.

In some embodiments, the medical device or system 109 is configured to be used for cardiac applications, such as generating an MCG. In other embodiments, the medical device or system 109 is used to sense an EMF associated with different parts of the body or for various diseases or conditions.

In some cases, the medical device or system 109 is employed for a prognostic method, such as predicting a likelihood of a subject developing a disease or condition; a diagnostic method, such as confirming a diagnosis or providing a diagnosis to a subject for a disease or condition; or a monitoring method, such as monitoring a progression of a disease or condition in a subject, monitoring an effectiveness of a therapy provided to a subject, or a combination thereof.

In some embodiments, the medical device or system 109 uses one or more OPMs in an n×n array (or grid) or alternative geometric configuration to collect magnetic field data at n discrete locations over a portion of a body of an individual (such as a chest area), which in some embodiments is digitized using pickup electronics and in some embodiments is connected to a computer for recording and displaying this data. It should be understood, however, that the medical device or system 109 is suitable for measuring an electromagnetic field associated with any type of tissue, for example, utilizing OPMs.

In some embodiments, the medical device or system 109 is configured to sense an EMF associated with, for example, a tissue, a body part, or an organ of an individual. In some embodiments, the medical device or system 109 comprises a mobile base unit and one or more EMF sensors.

In some embodiments, the medical device or system 109 comprises a mobile base unit, one or more EMF sensors, and a shield for shielding ambient electromagnetic noise. In some embodiments, a mobile base unit includes wheels or a track upon which the mobile base unit is moved on a surface.

FIG. 2 depicts an example platform architecture that may be deployed through an environment, such as platform 100 depicted in FIG. 1. The example platform architecture includes users 210, portals 220, PaaS services 230, external services 240, and API Gateway 250. As depicted, users 210 include global readers 212, site users 214, platform users 216, and patients 218. As depicted, portals 220, includes GRP 222, SRP 224, operator portal 226, internal portal 227, billing portal 228, and patient portal 229. In some embodiments, PaaS services 230 are deployed through as PaaS, such as Faraday. In some embodiments, the services 230 are implemented as microservices. As depicted, PaaS services 230 include user admin and authentication service 232, global reader service 233, site service 234, EHR integration service 235, signal processing service 236, machine-learning service 237, billing services 238, and internal service 239. In some embodiments, external services are services provided through third parties. As depicted, external services include SOS 242, S3 244, VPN 246, and EMR 248. In some embodiments, the API Gateway 250 is an exposed set of API endpoints that coordinates a set of calls to different microservices.

In some embodiments, global readers 212 include managed physicians with access to the GRP 222. In some embodiments, site users 214 include physicians, nurses, information technology (IT) personnel, administrators, and technicians with access to the SRP 224 or the operator portal 226. In some embodiments, platform users 216 include IT personnel, customer service personnel, developers, administrators, and billing personnel with access to the internal portal 227 or the billing portal 228. In some embodiments, patients 218 include patients with access to the patient portal 229.

In some embodiments, the user admin and authentication service 232 authenticates user credentials and provides access to other services in the API Gateway. In some embodiments, a user provides credentials (e.g., a username and password) to user admin and authentication service 232 when logging into the described platform. In some embodiments, the user admin and authentication service 232 returns a JSON Web Token (JWT) that allows the user to access other services. In some embodiments, the user admin and authentication service 232 stores user information, such as name, email, phone number, National Provider Identifier (NPI), routing and account numbers, authorization level, and so forth. In some embodiments, a user is allowed access to various portals and services by the user admin and authentication service 232 based on a respective user authorization level.

In some embodiments, the global reader service 233 provides services to the global reader portals 222. In some embodiments, global readers 212 have access to their own GRP 222. In some embodiments, cases from medical devices (e.g., CardioFlux) are routed to the appropriate specialty subset of readers within specified time slots, in the form of, for example, email or text, based on the reader's preference. The depicted architecture 200 allows sites to take the burden off their on-site physicians and outsource readings without providing readers with access to Patient Health Information. In some embodiments, scans are uniquely identified by a respective scan identifier and provide relevant site information. In some embodiments, based on volume in the queue of scans that need to be read, notifications are stratified to send cases based on how likely readers are to complete and submit interpretations in under a specified threshold (e.g., one hour). Interpretations may include scan quality assessment, diagnosis, and any other additional comments. In some embodiments, readers are provided in-depth trainings and certifications prior to being registered onto the platform and being allowed to read.

In some embodiments, the site service 234 provides patient information, scan interpretations and addendums received from global readers, access to customer service, an option to interface directly with global readers who have interpreted specific scans, and general support for SRP 224. In some embodiments, through the site service 234 user of sites can view all patient information that would otherwise be accessed directly from the EHR, with the addition of full dynamic reports for an integrated device, such as CardioFlux. In some embodiments, the site service 234 allows site administrators to assign levels of visibility based on user assignments that can be made for each new profile. User assignments may include physicians (e.g., with a full view of all patient information), technicians (e.g., that can access the operator portal), and information technology (e.g., that can submit service tickets on a device). In some embodiments, a users' visibility can be assigned and edited within an administrator view. In some embodiments, pushes to credential editing can be obtained (e.g., forgot my password).

In some embodiments, the EHR integration service 235 provides integration services for the employed PaaS. In some embodiments, the employed PaaS integrates with the integration service 235 to extract information in relation to a patient's use of a medical device. This information includes, but is not limited to, a patient's demographic, insurance, diagnoses, conditions and medical history. In some embodiments, this information is used and displayed throughout the applicable portals. In some embodiments, the employed PaaS integrates with the integration service 235 to push interpretations from Physicians back into the EHR. In some embodiments, information, such as interpretations, addendums, scan details and global reader identifying information is synthesized in a report. In some embodiments, such a report is generated directly within the EHR where physicians on-site with a device, such as CardioFlux, can access the information without adaptations or interruptions to their current workflow. In some embodiments, the employed PaaS integrates with the integration service 235 to allow on-site physicians to also order scans, such as MRIs, CTs, stress tests and custom scans, in tandem with hospital techs being able to operate associated medical devices with prefilled patient data fields. Such integration allows for devices to seamlessly function within new sites, with minimal training and outside consultation. In some embodiments, the employed PaaS integrates with the integration service 235 to populate information needed for filing insurance claims. At the end of the scan process, much of this information may been collected, but additional information, such as patient insurance information, provider and reader NPI information, reason for procedure, and other related procedures, can also be collected.

In some embodiments, the signal processing service 236 processes recording data sent from the medical devices, such as CardioFlux. In some embodiments, signal processing service 236 includes two pipelines—a processing pipeline and a signal previewing pipeline. In some embodiments, signal processing service 236 includes two additional libraries—an Interpolation Library and Quantification Library. In some embodiments, a signal previewing script runs in the Signal Previewing Pipeline—this component generates a preview of the cardiac signal after a threshold amount of data is collected, (e.g., after 60 seconds of data collection or a set number of bytes). In some embodiments, this preview is shown in the operator portal 226, which is discussed at length below. In some embodiments, a signal processing script runs in the signal processing pipeline. In some embodiments, this component generates the processed cardiac signal once a recording is complete and then quantifies the resulting magnetic field map. In some embodiments, the interpolation library, used by the Signal Processing Pipeline, handles interpolation of sensors in the final recording and is part of the signal quality determination process. In some embodiments, the parameter quantification library is used by the signal processing pipeline to handle the delineation of the T-wave and the quantification of the magnetic field map. In some embodiments, these components run on AWS Elastic Compute Cloud (EC2) instances and are deployed in Docker containers. In some embodiments, the Signal Processing Server is responsible for generating signal previews for the operator, generating the final processed signal, signal denoising, beat segmentation, cycle averaging, ensuring signal quality and magnetic field map generation, quantification and parameterization. In other device implementations, image/signal processing can be customized with a set of predefined protocols requested by device manufacturers.

In some embodiments, the machine-learning service 237 includes an artificial neural network (ANN). In some embodiments, the ANN is provided a goal to determine how well it can reconstruct the repolarization magnetic field time series images. In some embodiments, the ANN is trained and generates high-quality reconstruction of normal repolarization (ST-T) segments. The hypothesis follows as such: the higher the reconstruction error, the more likely the patient's repolarization period is indicative of abnormal activity. In some embodiments, the ANN is trained using samples and validated to minimize the reconstruction error. In some embodiments, to test the efficacy of the ANN, cases are presented that the network has not seen. Based on this method, a scoring method can be devised. In some embodiments, the scoring method ranges from 0 to 5, when 3 or above represents acute cardiac abnormalities.

In some embodiments, the billing service 238 automatically generates billing information. In some embodiments, EHR integration is integral to enable the billing functions of the PaaS, as most of the information that is needed to fill out insurance reimbursement forms can be found in hospital EHR systems. In some embodiments, this data is being collected throughout the workflow, and at completion of a scan, an internal billing analyst is presented with an auto-populated PDF form (e.g., CMS 1500 or UB-04) with patient demographic information, procedure codes and explanations, insurance information, and care provider information. In some embodiments, two forms are generated to receive reimbursement: one for the facility use of the device, and another for the physician read and interpretation of the scan data. In some embodiments, these claims are sent to the respective insurer (Center for Medicare & Medicaid Services, or other private insurer) and the claims process is tracked. In some embodiments, the internal billing analysts can add/modify information on this form, update the tracking process in the reimbursement lifecycle, and close any claims in the process. This service streamlines the billing process for the convenience of the care provider, institution, and the patient.

In some embodiments, the internal service 239 enables IT administration functions and handles overall user and site administration. For example, the internal service 239 may handle create, read, update, and delete (CRUD) functions for sites (hospitals), hospital admin users, and hospital usage statistics. In some embodiments, the internal service 239 is also used to manage the registration and verification of global readers used for the telehealth aspects of the PaaS Analytical Cloud.

In some embodiments, each of the portals 220 provides subsets of users' visibility to the data and/or requires access fields. In some embodiments, the GRP 222 is deployed separately for each managed physicians. In some embodiments, the GRP 222 provides notifications to physicians when scans are completed, a window to interpret these scans, and submission back to an original site. In some embodiments, through the GRP 222, physicians are able to modify the times they want to be notified through their active hours settings. For example, physicians can completely turn off their notifications or change how they receive these alerts (e.g., text or email). In addition, changes to username, password, email, and phone number can be made within the global reader “Settings” tab. In some embodiments, the GRP 222 provides a scan log for physicians that documents previous interpretations and addendums and allows for completion and submission of the documents. In some embodiments, each scan available in the GRP 222 has a unique scan identifier as well as the ordering physician's name, site and phone number for easy access of readers. In some embodiments, global readers are able to access customer service within their respective portal.

In some embodiments, the SRP 224 provides a list of patients that have taken a scan, such as a CardioFlux scan. In some embodiments, patient's information is auto-filled from information linking back to the EHR. In some embodiments, interpretations and addendums made from global readers can be viewed in the SRP 224. In some embodiments, users accessing SRP 224 can change their account settings, which allows them to alter their active hours and receive alerts based on the patients they created orders for. In some embodiments, physicians using their respective SRP 224 can request addendums from global readers on any previous scan that has been submitted. In some embodiments, the administrator view of the site portal provides the assignment of specific users; provides further information of site details, such as number of users, number of scans, and so forth; and helps others with credential information, such as forgot password and/or username. In some embodiments, the SRP 224 includes a customer service portal, where users can chat live with a representative, email from within the portal to track individual cases or directly call a support line. In some embodiments, a user can access the customer service portal and a self-service forum through the SRP 224. In some embodiments, a self-service center provides different levels of support ranging from the platform to the device for technicians needing it. In some embodiments, access to a SRP 224 and levels of visibility are assigned through a site administration portal. Based on the site administration's discretion, physicians, technicians, nurses, residents, and so forth can have access to the SRP 224.

In some embodiments, the operator portal 226 is accessed from a desktop that controls the physical device. In some embodiments, the operator portal 226 is used to collect, analyze, and display the magnetic field image data. From this portal operators can: activate and control medical devices, such as CardioFlux (including bed insertion and data acquisition modules), create or select a pre-existing patient (EHR integration will fill out patient information once initial fields are filled), collect magnetic field image data and send confirmed data to the site portal for processing and future use. In some embodiments, accepting magnetic field images as being of adequate quality automatically notifies the GRP 222 that there is a scan waiting to be read. In some embodiments, rejecting these images allows an operator to run the scan again or cancel the administration of the scan. In some embodiments, within the account settings, operators can also specify which alerts they wish to receive (e.g., physician orders scan, global reader rejects a scan due to quality, and so forth) and edit where they receive these alerts. In some embodiments, operators also have access to the customer service forum mentioned above. In some embodiments, operator visibility allows users to also access and create hardware tickets (for any issues with the physical device) that are directly posted.

In some embodiments, the internal portal 227 has users ranging from administrators, IT, customer service, and developers. In some embodiments, much like in the SRP 224, administrators can create accounts and assign users to different roles, which provide varying levels of access throughout the portal. In some embodiments, IT and customer service can view tickets that are filed and receive specific notifications to more closely monitor specific sites. Each ticket can be left unresolved, while it is being handled, or closed once there is a resolution from the user that filed the ticket. In some embodiments, tickets, customer complaints, calls and emails can also be tracked and viewed in Microsoft® Dynamics, as it is integrated with the customer service vendor's page. Developers can be flagged by customer service representatives based on the issue that needs to be solved. In some embodiments, the internal portal 227 provides analytics on each user that has been created, which portals they have access to, and critical statistics depending on the user base (e.g., average time per scan for global readers, monthly scans for site portals, number of completed claims for billing portals, patient dialogue for patient portals, and so forth).

In some embodiments, internal billing analysts have access to a separate billing portal 228. In some embodiments, the billing portal 228 includes information on each claim that an individual has completed. In some embodiments, much like the scan log, the billing portal 228 includes a claim log where relevant information regarding a patient and their provider are provided. In some embodiments, analysts can change the status of each claim as it is processed. Moreover, as with global readers, billing analysts can control which notifications they receive (based on each claim update) and how they receive them (phone/text). For example, based on each set of unique codes, analysts can choose exactly which follow-up information is required to most effectively file follow-ups to claims. In some embodiments, draft templates for relevant follow-ups can be found under “templates” in addition to best practices to submit each claim. This information can also be found in the customer service tab, with the self-service forum. This information, including general portal features and FAQs, can also be found here. In some embodiments, the billing portal 228 displays billing analytics as they pertain to successful cases, pending cases, rejected cases, and so forth.

In some embodiments, when a patient has taken a scan from a monitored medical device, such as described above, they are given a unique set of credentials (e.g., based on a scan identifier) to view all follow-ups in reference to their claim. In some embodiments, the patient portal 229 provides these patients updates in the status of the claim that are, for example, filed on the hospital's behalf. In some embodiments, in account settings, patients can view and select alerts (e.g., submissions, re-submissions, acceptances, and so forth). In some embodiments, through the patient portal 229, patients can choose to interact directly with a customer support forum, which may include self-service search, live chat with representatives, email and call.

### EMF Sensing Devices and Systems

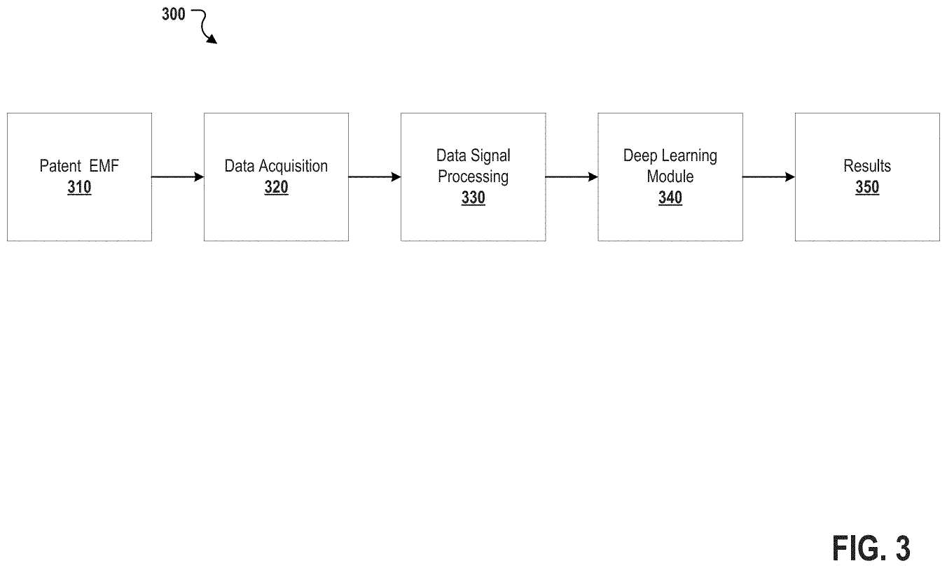

FIG. 3 depicts a schematic representation of an exemplary medical device or system 300 for sensing and/or analyzing an EMF. In some embodiments, medical device or system 300 can be deployed in an environment, such as platform 100, and include medical device or system 109 of FIG. 1. It should be also understood that any medical device or system is suitable for use with the platforms described herein including and not limited to medical imaging and medical monitoring systems. Generally, any medical device or system that receives, generates, or senses medical data from an individual is suitable for use in addition to or in place of the medical device or system 300 in various embodiments of the platforms described herein.

As shown in FIG. 3, an EMF 310, which is associated with an individual (e.g., an EMF generated by a current traveling through myocardium), is acquired from the EMF sensor or sensors 320 (e.g., a sensor array). The data is then processed, optionally filtered and analyzed by a signal processing module 330. A signal processing module 330 in some embodiments removes noise if any from the sensed EMF signal and extracts information from the data. The processed data is then fed into the deep learning module 340 that, in some embodiments, includes dilated convolutional neural networks. The deep learning module detects, for example, ischemia and localizes to a particular region in an organ and provides these as results 350.

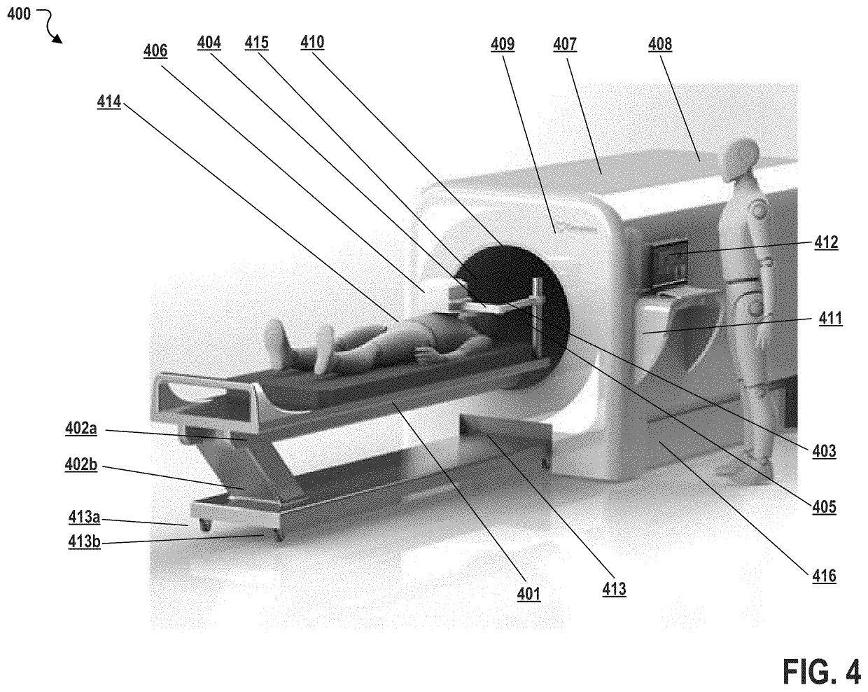

FIG. 4 depicts an exemplary embodiment of a medical device or system 400 for sensing an EMF. In some embodiments, the medical device or system 400 can be deployed in an environment, such as platform 100, as the medical device or system 109 of FIG. 1. As depicted, medical device or system 400 includes a shield 407 and a sensor 406 (such as an optically pumped magnetometer). A shield 407 may comprise an open end 409 and a closed end 408. In some cases, the open end 409 is positioned adjacent to the closed end 408. In some cases, the open end 409 is positioned opposite to the closed end 408. A shield 407 may comprise one or more openings. Such one or more openings in some embodiments is configured to receive at least a portion of a base unit 401, at least a portion of an individual 414, at least a portion of a sensor 406, or any combination thereof. For example, a shield 407 may comprise an opening, such as a recess opening 413 configured to receive a portion of a base unit 401. A shield 407 may comprise an opening 415 configured to receive at least a portion of a base unit 401, at least a portion of an individual 414, at least a portion of a sensor 406, or any combination thereof. A shield 407 may comprise an inner surface 410. In some cases, an inner surface may comprise a coating. An inner surface 410 of a shield 407 may define an inner volume of a shield. An inner volume of a shield 407 in some embodiments is a volume into which a portion of an individual, a portion of a sensor, a portion of a base unit, or any combination thereof in some embodiments is received. A shield may comprise a portion 416 configured to store a component of a device for sensing an EMF, such as an electronic driver. A portion may comprise a drawer, a shelf, a cabinet, a compartment, or a section of a shield. A portion in some embodiments is positioned on a side portion of a shield. A portion in some embodiments is positioned on a bottom portion of a shield.

In some cases, a device or system for sensing an EMF 400 as described herein may comprise a base unit 401. In some cases, a device for sensing an EMF as described herein in some embodiments is operatively coupled with a base unit 401. In some cases, a shield 407 is configured to receive a portion of a base unit 401, such as, for example, a recess opening of a shield 407 is configured to receive a base portion of a base unit, as shown in FIG. 4. In some embodiments, a base unit is attachable to a device for sensing an EMF, such as attachable to a shield. In some cases, a base unit 401 is operatively connected to a device for sensing an EMF.

A base unit 401, in some embodiments, is configured as a stationary base unit. A base unit in some embodiments is configured as a mobile base unit. In some cases, a shield in some embodiments is movable relative to a base unit. In some cases, a base unit in some embodiments is movable relative to a shield. In some cases, a base unit and a shield in some embodiments are movable relative to one another.

In some cases, a base unit 401 in some embodiments is configured as a movable base unit, such as shown in FIG. 4. A movable base unit in some embodiments is configured to move in one or more degrees of freedom. In some cases, a movable base unit in some embodiments is configured to move along an x axis, a y axis, a z axis, or any combination thereof. A movable base unit may comprise one or more rotating elements such as a wheel (413a, 413b), a roller, a conveyor belt, or any combination thereof configured to provide movement of a base unit. In some cases, a base unit 401 comprises one rotating element. In some cases, a base unit may comprise two rotating elements. In some cases, a base unit 401 comprises three rotating elements. In some cases, a base unit 401 may comprise four rotating elements. In some cases, a base unit 401 comprises more than four rotating elements. In some cases, a rotating element is positioned at one or both ends of a base unit. In some cases, a base unit 401 may comprise a non-rotating element configured to be received into a track or channel such that the base unit is movable along the track or channel. In some cases, the track or channel in some embodiments is positioned adjacent to a shield, such that the base unit 401 in some embodiments is movable towards, away, or both from the shield. A base unit 401 may comprise one or more pivots (402a, 402b). In some cases, a base unit may comprise one pivot. In some cases, a base unit may comprise two pivots. In some cases, a base unit 401 may comprise more than two pivots. A pivot in some embodiments is configured to permit movement of a base unit such as to accommodate an individual being positioned onto a base unit. A pivot in some embodiments is configured to permit movement of a base unit such as to position the base unit within an inner volume of a shield. A pivot in some embodiments is configured to provide movement to the base unit having one or more degrees of freedom.

In some cases, a sensor 406 in some embodiments is operatively coupled to an arm 403. An arm in some embodiments is a movable arm, such as movable in at least one degree of freedom. An arm 403 may comprise a joint 404 configured to provide movement to the arm. In some cases, an arm may comprise more than one joint. In some cases, an arm may comprise two joints. An arm in some embodiments is operatively coupled to a sensor and to a base unit, such as shown in FIG. 4. An arm in some embodiments is operatively coupled to a base unit by a beam 405. A beam in some embodiments is attached to a base unit and to the arm.

In some cases, a device for sensing an EMF 400 as described herein may comprise a computer processor 412, as shown in FIG. 4. A computer processor 412 may comprise a graphical user interface. A computer processor 412 may comprise a touchscreen. A medical device for sensing an EMF 400 may comprise a stand 411 configured to receive a computer processor. A stand in some embodiments is positioned adjacent a shield 47 or a base unit 401. A stand 411 in some embodiments is integral to or attachable to a shield or a base unit of a device for sensing an EMF.

The devices and systems as described herein may have enhanced clinical utility, wherein biomagnetic measurements can be made from a mobile unit. The devices and systems as described herein may comprise a mobile unit (i.e., cart structure), such as a mobile unit comprising at least 2 wheels. In some cases, a mobile unit may comprise 4 wheels. In some cases, the device may have an extensible arm, at the end of which a sensor array may be housed. Any type of OPM may be used. An OPM may be integrated in the magnetocardiograph in an n-channel array. In some cases, the device may include a compartment and a tabletop to house electronics, a computer interface, and a power supply, and in others it may involve a separate unit to house these components, connected to the first component by wiring. In some cases, the device may require a power supply via an electrical outlet. Standard operating procedure may include extending a device's arm and lowering a base of a sensor unit to a position, such as a position that may be within 2 centimeters adjacent a skin surface of a subject (such as a subject's chest, head, or other region of interest). The device may be turned on and may be calibrated using a software application that may be provided with the device or provided separately. A biomagnetic signal of interest may be displayed and recorded for immediate or later analysis.

An operation of a device or system may be controlled using a software User Interface (UI). In some cases, a software UI may be installed on site, on a provided accessory computer. The use of the device may be prescribed by a medical professional such as a physician to determine more information regarding a subject's condition. Within the software user interface, User preferences and acquisition parameters may be chosen, including a sampling rate and an axis operation of the device or system. From the software user interface, magnetic field signals from a subject, such as signals corresponding to a subject's heart, can be displayed and can be saved to a file. The device or system may be used to measure cardiac electrical activity, creating waveforms similar to electrocardiograph recordings which may demonstrate points of interest in a cardiac cycle.

A device or system may be constructed to overcome tradeoffs associated with older SQUID devices to maximize clinical utility, while remaining cost-effective and technician-friendly. A device or system may present no physical risk to a subject and may be an adjunctive tool employed in addition to a second medical procedure or clinical measurement in order to aid a physician to provide more detailed information regarding a subject's condition. These inventions are the first of their kind using optically pumped magnetometers for measurements of biomagnetic measurements. A device or system as described herein is the first example of OPMs used in a compact shield based design. A device or system as described herein may be the first entirely self-contained biomagnetic detection system that utilizes this compact shield design. A device or system as described herein is the first example of a mobile cart and bedside deployable unit for biomagnetic measurements.

Traditional OPMs that have a desired level of sensitivity for biomagnetic measurements are understood to have a dynamic range which necessarily limits their use to low magnetic field environments, wherein ambient noise is generally less than about 100 nanotesla. The earth's magnetic field is naturally present everywhere on earth, and the amplitude is about 50 microtesla (about 500 times greater than the ceiling of operation of a device as described herein).

To combat ambient noise, some embodiments of the devices and systems described herein provide an electromagnetic shield comprising a metal alloy (e.g., permalloy or mumetal), which when annealed in a hydrogen furnace typically have exceptionally high magnetic permeability. When formed into a shielding barrier or chamber, the permeable alloy absorbs magnetic field signals and provides a pathway for the magnetic signals to travel along (i.e., on the surface of or within the body of the alloy) so as to shield the embodiments of the devices and systems that include these shields.

In some embodiments, a device or system as described herein comprises a shield in the form of a large chamber configured to minimize interior magnetic fields within the chamber, and in some embodiments is constructed with one closed end and one open end. The closed end may take the form of a flat, conical, or domed endcap. The shield in some embodiments is housed in a larger shrouded structure, and due to the size requirements for adequate shielding, the total device length in some embodiments is at minimum about 1.5 meters (m) in length, with a bore opening (or an internal opening diameter) of about 0.8 m.

In order to insert a subject into a shield, a base unit (such as a bed platform) may be used upon which the subject may be positioned. During device use, a flexible jointed arm with x-y-z translational movement (may be able to occupy any point within a semicircle defined by total arm length at extension) may be used to position an array of n-optically pumped magnetometers in a wide range of geometries on or proximally above a portion of a subject (such as a subject's chest, head, or other organ) using a set standard operating procedure based on an organ of interest, a condition or disease of interest, or a combination thereof. After this point, the sensor array may be turned on and at least a portion of the subject, at least a portion of the base unit (i.e., bed platform), or a combination thereof may be slid into the shield. Using a provided computer application, fast calibration of the sensors may occur, and then the magnetic field of the organ of interest can be displayed, can be recorded, or a combination thereof for immediate or later analysis. Electronic drivers for the sensors may be housed either underneath the shield portion of the device, or may be housed in an adjacent cart with computer control. The system may also involve a touch screen computer interface (such as a graphical user interface) housed on a side of the device itself, or on said adjacent cart.

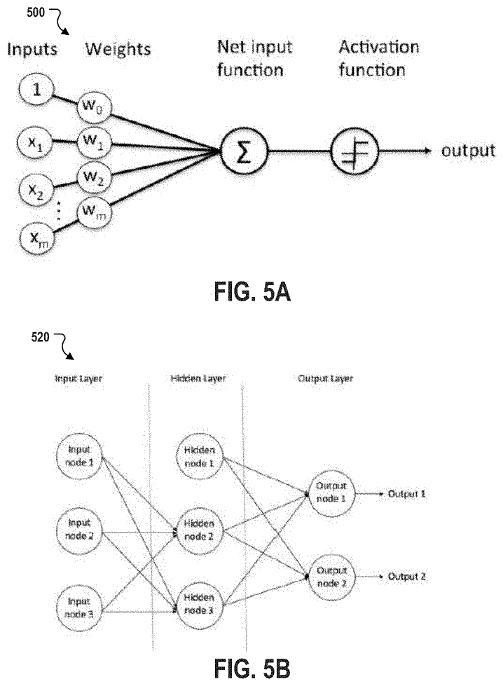

In some embodiments, an ANN, such as the ANN depicted in FIG. 5A, may be employed within the machine-learning service 237 of FIG. 2 comprised of a series of layers termed “neurons.” FIG. 5A depicts typical neuron 500 in an ANN. As illustrated in FIG. 5B, in embodiments of ANNs 520, there is an input layer to which data is presented; one or more internal, or “hidden,” layers; and an output layer. A neuron may be connected to neurons in other layers via connections that have weights, which are parameters that control the strength of the connection. The number of neurons in each layer may be related to the complexity of the problem to be solved. The minimum number of neurons required in a layer may be determined by the problem complexity, and the maximum number may be limited by the ability of the neural network to generalize. The input neurons may receive data from data being presented and transmit that data to the first hidden layer through connections' weights, which are modified during training. The first hidden layer may process the data and transmit its result to the next layer through a second set of weighted connections. Each subsequent layer may “pool” the results from the previous layers into more complex relationships. In addition, whereas conventional software programs require writing specific instructions to perform a function, neural networks are programmed by training them with a known sample set and allowing them to modify themselves during (and after) training so as to provide a desired output such as an output value. After training, when a neural network is presented with new input data, it is configured to generalize what was “learned” during training and apply what was learned from training to the new previously unseen input data in order to generate an output associated with that input.

In some embodiments of a machine learning software module as described herein, a machine learning software module comprises a neural network such as a deep convolutional neural network. In some embodiments in which a convolutional neural network is used, the network is constructed with any number of convolutional layers, dilated layers or fully connected layers. In some embodiments, the number of convolutional layers is between 1-10 and the dilated layers between 0-10. In some embodiments, the number of convolutional layers is between 1-10 and the fully connected layers between 0-10.

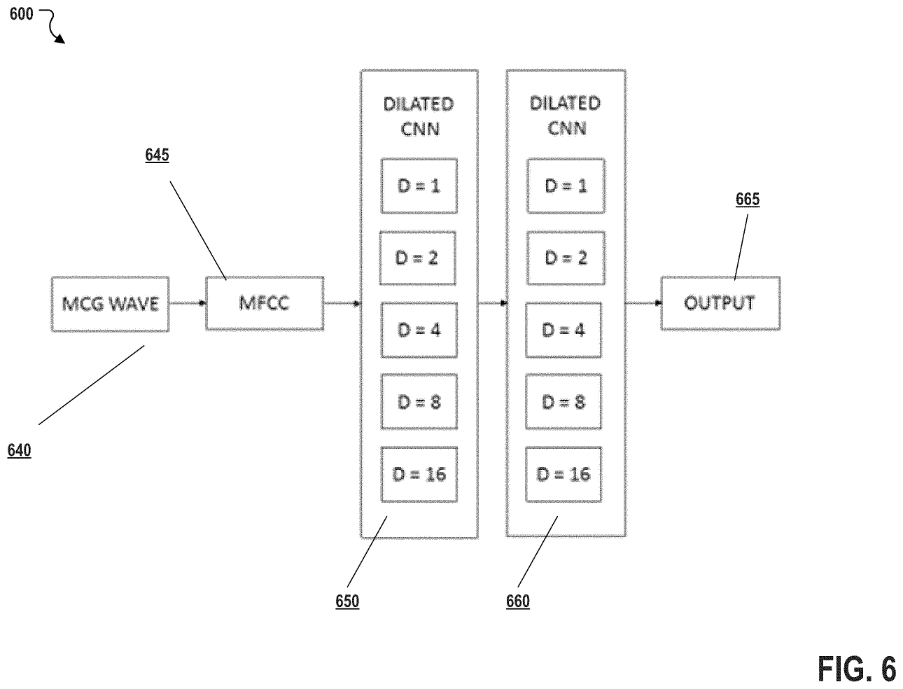

FIG. 6 depicts a flow chart 600 representing the architecture of an exemplary embodiment of a machine learning software module, which may be employed within the machine-learning service 237 of FIG. 2. In this exemplary embodiment, raw EMF 640 of the individual is used to extract the MFCC features 645 which are fed into the deep learning module. The machine learning software module comprises two blocks of Dilated Convolutional neural networks 650, 660. Each block has 5 dilated convolution layers with dilation rates D=1, 2, 4, 8, 16. The number of blocks and the number of layers in each block can increase or decrease, so it is not limited to the configuration portrayed in FIG. 6.

a. Training Phase

A machine learning software module as described herein is configured to undergo at least one training phase wherein the machine learning software module is trained to carry out one or more tasks including data extraction, data analysis, and generation of output 665.

In some embodiments of the software application described herein, the software application comprises a training module that trains the machine learning software module. The training module is configured to provide training data to the machine learning software module, said training data comprising, for example, EMF measurements and the corresponding abnormality data. In additional embodiments, said training data is comprised of simulated EMF data with corresponding simulated abnormality data. In some embodiments of a machine learning software module described herein, a machine learning software module utilizes automatic statistical analysis of data in order to determine which features to extract and/or analyze from an EMF measurement. In some of these embodiments, the machine learning software module determines which features to extract and/or analyze from an EMF based on the training that the machine learning software module receives.

In some embodiments, a machine learning software module is trained using a data set and a target in a manner that might be described as supervised learning. In these embodiments, the data set is conventionally divided into a training set, a test set, and, in some cases, a validation set. A target is specified that contains the correct classification of each input value in the data set. For example, a set of EMF data from one or more individuals is repeatedly presented to the machine learning software module, and for each sample presented during training, the output generated by the machine learning software module is compared with the desired target. The difference between the target and the set of input samples is calculated, and the machine learning software module is modified to cause the output to more closely approximate the desired target value. In some embodiments, a back-propagation algorithm is utilized to cause the output to more closely approximate the desired target value. After a large number of training iterations, the machine learning software module output will closely match the desired target for each sample in the input training set. Subsequently, when new input data, not used during training, is presented to the machine learning software module, it may generate an output classification value indicating which of the categories the new sample is most likely to fall into. The machine learning software module is said to be able to “generalize” from its training to new, previously unseen input samples. This feature of a machine learning software module allows it to be used to classify almost any input data which has a mathematically formulatable relationship to the category to which it should be assigned.

In some embodiments of the machine learning software module described herein, the machine learning software module utilizes an individual learning model. An individual learning model is based on the machine learning software module having trained on data from a single individual and thus, a machine learning software module that utilizes an individual learning model is configured to be used on a single individual on whose data it trained.

In some embodiments of the machine training software module described herein, the machine training software module utilizes a global training model. A global training model is based on the machine training software module having trained on data from multiple individuals and thus, a machine training software module that utilizes a global training model is configured to be used on multiple patients/individuals.

In some embodiments of the machine training software module described herein, the machine training software module utilizes a simulated training model. A simulated training model is based on the machine training software module having trained on data from simulated EMF measurements. A machine training software module that utilizes a simulated training model is configured to be used on multiple patients/individuals.

In some embodiments, the use of training models changes as the availability of EMF data changes. For instance, a simulated training model may be used if there are insufficient quantities of appropriate patient data available for training the machine training software module to a desired accuracy. This may be particularly true in the early days of implementation, as few appropriate EMF measurements with associated abnormalities may be available initially. As additional data becomes available, the training model can change to a global or individual model. In some embodiments, a mixture of training models may be used to train the machine training software module. For example, a simulated and global training model may be used, utilizing a mixture of multiple patients' data and simulated data to meet training data requirements.

Unsupervised learning is used, in some embodiments, to train a machine training software module to use input data such as, for example, EMF data and output, for example, a diagnosis or abnormality. Unsupervised learning, in some embodiments, includes feature extraction which is performed by the machine learning software module on the input data. Extracted features may be used for visualization, for classification, for subsequent supervised training, and more generally for representing the input for subsequent storage or analysis. In some cases, each training case may consist of a plurality of EMF data.

Machine learning software modules that are commonly used for unsupervised training include k-means clustering, mixtures of multinomial distributions, affinity propagation, discrete factor analysis, hidden Markov models, Boltzmann machines, restricted Boltzmann machines, autoencoders, convolutional autoencoders, recurrent neural network autoencoders, and long short-term memory autoencoders. While there are many unsupervised learning models, they all have in common that, for training, they require a training set consisting of biological sequences, without associated labels.

A machine learning software module may include a training phase and a prediction phase. The training phase is typically provided with data in order to train the machine learning algorithm. Non-limiting examples of types of data inputted into a machine learning software module for the purposes of training include medical image data, clinical data (e.g., from a health record), encoded data, encoded features, or metrics derived from an electromagnetic field. Data that is inputted into the machine learning software module is used, in some embodiments, to construct a hypothesis function to determine the presence of an abnormality. In some embodiments, a machine learning software module is configured to determine if the outcome of the hypothesis function was achieved and based on that analysis make a determination with respect to the data upon which the hypothesis function was constructed. That is, the outcome tends to either reinforce the hypothesis function with respect to the data upon which the hypothesis functions was constructed or contradict the hypothesis function with respect to the data upon which the hypothesis function was constructed. In these embodiments, depending on how close the outcome tends to be to an outcome determined by the hypothesis function, the machine learning algorithm will either adopt, adjust, or abandon the hypothesis function with respect to the data upon which the hypothesis function was constructed. As such, the machine learning algorithm described herein dynamically learns through the training phase what characteristics of an input (e.g., data) are most predictive in determining whether the features of a patient EMF display any abnormality.

For example, a machine learning software module is provided with data on which to train so that it, for example, is able to determine the most salient features of a received EMF data to operate on. The machine learning software modules described herein train as to how to analyze the EMF data, rather than analyzing the EMF data using pre-defined instructions. As such, the machine learning software modules described herein dynamically learn through training what characteristics of an input signal are most predictive in determining whether the features of an EMF display any abnormality.

In some embodiments, the machine learning software module is trained by repeatedly presenting the machine learning software module with EMF data along with, for example, abnormality data. The term “abnormality data” is meant to comprise data concerning the existence or non-existence of an abnormality in an organ, tissue, body, or portion thereof. Any disease, disorder or condition associated with the abnormality is included in the abnormality data if available. For example, information concerning a subject displaying symptoms of hypertension, ischemia or shortness of breath is included as abnormality data. Information concerning a subject's lack of any irregular health condition is also included as abnormality data. In the case where EMF data is generated by computer simulation, the abnormality data may be used as additional data being used to simulate the organ, tissue, body, or portion thereof. In some embodiments, more than one abnormality is included in the abnormality data. In additional embodiments, more than one condition, disease or disorder is included in the abnormality data.

In some embodiments, training begins when the machine learning software module is given EMF data and asked to determine the presence of an abnormality. The predicted abnormality is then compared to the true abnormality data that corresponds to the EMF data. An optimization technique such as gradient descent and backpropagation is used to update the weights in each layer of the machine learning software module so as to produce closer agreement between the abnormality probability predicted by the machine learning software module, and the presence of the abnormality. This process is repeated with new EMF data and abnormality data until the accuracy of the network has reached the desired level. In some embodiments, the abnormality data additionally comprises the type and location of the abnormality. For example, the abnormality data may indicate that an abnormality is present, and that said abnormality is an ischemia of the left ventricle of the heart. In this case, training begins when the machine learning software module is given the corresponding EMF data and asked to determine the type and location of the abnormality. An optimization technique is used to update the weights in each layer of the machine learning software module so as to produce closer agreement between the abnormality data predicted by the machine learning software module, and the true abnormality data. This process is repeated with new EMF data and abnormality data until the accuracy of the network has reached the desired level. In some embodiments, the abnormality data additionally comprises a known resulting or related disease, disorder or condition associated with an identified abnormality. For example, the abnormality data may indicate that the subject possesses an atrial flutter and arterial coronary disease. In cases such as this, training begins when the machine learning software module is given the corresponding EMF data and asked to determine the presence of a condition, disorder or disease. The output data is then compared to the true abnormality data that corresponds to the EMF data. An optimization technique is used to update the weights in each layer of the machine learning software module so as to produce closer agreement between the abnormality probability predicted by the machine learning software module, and the actual abnormality. This process is repeated with new EMF data and abnormality data until the accuracy of the network has reached the desired level. Following training with the appropriate abnormality data given above, the machine learning module is able to analyze an EMF measurement and determine the presence of an abnormality, the type and location of said abnormality and the conditions associated with such.

In some embodiments of the machine learning software modules described herein, the machine learning software module receives EMF data and directly determines the abnormality probability of the subject, wherein the abnormality probability comprises the probability that the EMF measurement is associated with the abnormality of the subject.

In some embodiments, the machine learning software module is trained on a single continuous EMF measurement with corresponding abnormality data over a period of time. This can greatly increase the amount of training data available to train a machine learning software module. For example, in an EMF recording consisting of N continuous 10-second segments with accompanying abnormality data, one can generate at least N*N pairs of such segments to train on.

In some embodiments, an individual's abnormality data is inputted by the individual of the system. In some embodiments, an individual's abnormality data is inputted by an entity other than the individual. In some embodiments, the entity can be a healthcare provider, healthcare professional, family member or acquaintance. In additional embodiments, the entity can be the instantly described system, device or an additional system that analyzes EMF measurements and provides data pertaining to physiological abnormalities.

In some embodiments, a strategy for the collection of training data is provided to ensure that the EMF measurements represent a wide range of conditions so as to provide a broad training data set for the machine learning software module. For example, a prescribed number of measurements during a set period of time may be required as a section of a training data set. Additionally these measurements can be prescribed as having a set amount of time between measurements. In some embodiments, EMF measurements taken with variations in a subject's physical state may be included in the training data set. Examples of physical states include accelerated heart rate and enhanced brain signaling. Additional examples include the analysis of a subject's EMF data under the influence of medication or during the course of medical treatment.

In some embodiments, training data may be generated by extracting random overlapping segments of EMF measurements performed by the subject. In some embodiments, training examples can be provided by measurement recordings, models or algorithms that are independent of the subject. Any mixture or ratio of subject and non-subject training measurements can be used to train the system. For example, a network may be trained using 5 EMF segments extracted from a subject's measurements, and 15,000 EMF segments taken from another subject's recordings. Training data can be acquired using two different methods. The first method is to directly measure the EMF measurements over a subject's chest. The second method involves creating an accurate electro-anatomical model of the heart. This electro-anatomical model can be used to generate EMF measurements of both healthy and diseased subjects. The measurements are acquired by applying the Biot-Savart Law. This calculates the magnetic field vector at a given point in space, caused by a specific movement of current. After the EMF measurements have been acquired or calculated, they are fed into the network with a classification label, describing both the presence and location of diseased tissue.

In general, a machine learning algorithm is trained using a large patient database of medical image and/or clinical data and/or encoded data from one or more EMF measurements and/or any features or metrics computed from the above said data with the corresponding ground-truth values. The training phase constructs a transformation function for predicting probability of an abnormality in an unknown patient's organ, tissue, body, or portion thereof by using the medical image and/or clinical data and/or encoded data from the one or more EMF measurements and/or any features or metrics computed from the above said data of the unknown patient. The machine learning algorithm dynamically learns through training what characteristics of an input signal are most predictive in determining whether the features of a patient EMF data display any abnormality. A prediction phase uses the constructed and optimized transformation function from the training phase to predict the probability of an abnormality in an unknown patient's organ, tissue, body, or portion thereof by using the medical image and/or clinical data and/or encoded data from the one or more EMF measurements and/or any features or metrics computed from the above said data of the unknown patient.

b. Prediction Phase

Following training, the machine learning algorithm is used to determine, for example, the presence or absence of an abnormality on which the system was trained using the prediction phase. With appropriate training data, the system can identify the location and type of an abnormality, and present conditions associated with such abnormality. For example, an EMF measurement is taken of a subject's brain and appropriate data derived from the EMF measurement is submitted for analysis to a system using the described trained machine learning algorithm. In these embodiments, a machine learning software algorithm detects an abnormality associated with epilepsy. In some embodiments, the machine learning algorithm further localizes an anatomical region associated with an abnormality such as, for example, localizing an area of the brain of an individual associated with epilepsy in the individual based on an EMF measurement of an individual.

An additional example, a subject is known to possess arterial ischemia and has EMF measurements recorded before and after treatment with a medication. The medical image and/or clinical data and/or encoded data from the EMF measurements and/or features and/or metrics derived from the said data are submitted for analysis to a system using the described trained machine learning algorithm in order to determine the effectiveness of the medication on abnormal blood flow using the prediction phase.

The prediction phase uses the constructed and optimized hypothesis function from the training phase to predict the probability of an abnormality in an unknown patient's organ, tissue, body, or portion thereof by using the medical image and/or clinical data and/or encoded data from the EMF measurements and/or any features or metrics computed from the above said data of the unknown individual.

In some embodiments, in the prediction phase, the machine learning software module can be used to analyze data derived from its EMF measurement independent of any system or device described herein. In these instances, the new data recording may provide a longer signal window than that required for determining the presence of a subject's abnormality. In some embodiments, the longer signal can be cut to an appropriate size, for example 10 seconds, and then can be used in the prediction phase to predict the probability of an abnormality of the new patient data.

In some embodiments, a probability threshold can be used in conjunction with a final probability to determine whether or not a given recording matches the trained abnormality. In some embodiments, the probability threshold is used to tune the sensitivity of the trained network. For example, the probability threshold can be 1%, 2%, 5%, 10%, 15%, 20%, 25%, 30%, 35%, 40%, 45%, 50%, 55%, 60%, 65%, 70%, 75%, 80%, 85%, 90%, 95%, 98% or 99%. In some embodiments, the probability threshold is adjusted if the accuracy, sensitivity or specificity falls below a predefined adjustment threshold. In some embodiments, the adjustment threshold is used to determine the parameters of the training period. For example, if the accuracy of the probability threshold falls below the adjustment threshold, the system can extend the training period and/or require additional measurements and/or abnormality data. In some embodiments, additional measurements and/or abnormality data can be included into the training data. In some embodiments, additional measurements and/or abnormality data can be used to refine the training data set.

### Input Data

As described herein, a machine learning software module is typically provided with data (input) in order to train the machine learning software module as to how to analyze an EMF to determine, for example, the presence of an abnormality. Input data is also used by a machine learning software module to generate an output.

An input to a machine learning algorithm as described herein, in some embodiments, is data transmitted to the machine learning algorithm by a device or a system which includes an EMF sensor. In some embodiments of the devices, systems, software, and methods described herein, data that is received by a machine learning algorithm software module from an electromagnetic sensor as an input may comprise EMF data expressed in a standard unit of measurement such as, for example, Tesla.

In some embodiments, sensed EMF data comprises an overall or total EMF generated by a body of an individual based on numerous different currents generated by the body of the individual. That is, in some embodiments, one or more EMF sensors sense an EMF that comprises an EMF associated with an entire individual and is not specific to a single organ, tissue, body, or portion thereof. Likewise, in some embodiments, an EMF is sensed from an individual that is associated with a portion of the individual, but not specific to a single organ, tissue, body, or portion thereof.

In some embodiments, sensed EMF data comprises an EMF that is in proximity to an individual or a portion of the body of the individual and comprises an EMF associated with a single organ, organ system, or tissue. For example, in some embodiments, one or more EMF sensors are positioned in proximity to a chest of an individual and sense an EMF associated with a heart of the individual. For example, in some embodiments, one or more EMF sensors are positioned in proximity to a head of an individual and sense an EMF associated with a brain of the individual. For example, in some embodiments, one or more EMF sensors are positioned in proximity to a chest of an individual and sense an EMF associated with a cardio-pulmonary system (i.e., the heart and lungs).

In some embodiments, a machine learning software module is configured to receive an encoded length of EMF data as an input and to determine the window length of the input data. For example, an input to a machine learning software module in some embodiments described herein is 100 seconds of encoded EMF data, and the machine learning software module selects a 10 second segment within the 100 second data sample for examination. In some embodiments, the input is segmented into multiple inputs, any number of which is analyzed independently. Any number of these analyses may be used to determine the final output.

In some embodiments, a device, system, or method as described herein is configured to sense and/or receive data comprising data associated with an individual. Data is sensed, in some embodiments, by an electromagnetic field sensor that is a component of a device, system, or method described herein. Data is received, in some embodiments, by transmission of data to a software algorithm as described herein by a source other than an EMF that is a component of a device, system, or method that also includes the software algorithm. That is, data, in some embodiments, is received from a source remote from the device, system, or method that includes the software algorithm. In some embodiments, data that is received comprises stored data. In some embodiments, data that is received comprises data that is generated by a software module. In general, sensed and/or received data comprises an input to a machine learning algorithm as described herein. An input is used to train a machine learning algorithm and/or is used by the machine learning algorithm to carry out an analysis or prediction.

Data as described herein comprises EMF data as well as other information associated with an individual. Non-limiting examples of data used as an input for a machine learning algorithm as described herein include a medical record (e.g., an electronic health record), a diagnosis, a lab value, a vital sign, a prognosis, an electrocardiogram, a radiology image (including ultrasound, CT scan, MM, and X-ray), an electroencephalogram, and a pathology report. In some embodiments, two or more different types of data are combined and/or correlated by the software algorithms described herein.

EMF data, in some embodiments, is used to generate other types of data that are used by the software algorithms described herein. For example, EMF data, in some embodiments, is used to generate medical image data which, in some embodiments, is achieved using Magnetic Field Maps (MFM). In some embodiments, EMF data is used to generate medical image data using Pseudo-Current Density (PCD) maps. In some embodiments, EMF data is used to generate medical data using Spatio-Temporal Activation Graphs (STAG).

EMF data, in some embodiments, is used to generate clinical data such as MCG, MEG and MGG measurements.

In some embodiments, input to a software algorithm as described herein comprises EMF data which is encoded into some other form of data and the features or metrics computed from the encoded data such as, for example, MFCC.

In some embodiments, input to a software algorithm as described herein is generated by a computer. For example, in some embodiments, an input to a software algorithm as described herein comprises data generated by computer simulation. In some embodiments, a computer simulation generates an image or other representation of an organ or other tissue (including skin, bone, and blood). In some embodiments, a computer simulation generates an image or representation of a flow of a fluid such as, for example, blood, lymph, or bile. In some embodiments, a computer simulation generates an image or representation of a flow of an electric current. Non-limiting examples of additional inputs generated by a computer simulation include a medical record (e.g., an electronic health record), a diagnosis, a lab value, a vital sign, a prognosis, an electrocardiogram, a radiology image (including ultrasound, CT scan, MRI, and X-ray), an electroencephalogram, and a pathology report.

### Data Filtering

In some embodiments of the devices, systems, software, and methods described herein, data that is received by a machine learning algorithm software module from an electromagnetic sensor as an input may comprise EMF data that has been filtered and or modified. In some embodiments, filtering comprises a removal of noise or artifacts from a sensed electromagnetic field data. Artifacts or noise may comprise, for example, ambient electromagnetic signals that are sensed together with electromagnetic data sensed from an individual.

In some embodiments of the devices, systems, software, and methods described herein, sensed EMF data is filtered prior to and/or after transmission of said data to a processor. Filtering of sensed EMF data may, for example, comprise the removal of ambient signal noise from a sensed EMF data. Signal noise may, for example, comprise ambient EMF data generated by, for example, electronic devices, the earth's magnetosphere, electrical grids, or other individuals (i.e., not individuals whose EMF data is being targeted).

In some embodiments, sensed EMF data is converted to another form of data or signal which then undergoes a signal filtering process. In some embodiments, a device or system includes a processor including software that is configured to convert sensed EMF data to another form of data or signal. The process of converting sensed EMF data to another form of data or signal typically comprises an encoding process, wherein a first form of data is converted into a second form of data or signal.

In some embodiments, sensed EMF data is encoded into an audio signal which undergoes a filtering process. In some embodiments, sensed EMF data is encoded into an audio signal or alternatively, a signal having the morphology of an audio signal.

In some embodiments, sensed EMF data is encoded into an audio signal which is further processed into a Mel-Frequency Cepstrum from which one or more Mel-Frequency Cepstrum Coefficients (“MFCC”) are derived. Mel-Frequency Cepstrum (“MFC”) represents a short term power spectrum of a sound. It is based on a linear cosine transform of a log power spectrum on a nonlinear mel scale of frequency. Mel-frequency cepstral coefficients (“MFCCs”) collectively make up an MFC. These are derived from a type of cepstral representation of the audio. In MFC, frequency bands are equally spaced on the mel-scale as compared to the linearly-spaced frequency bands used in the normal cepstrum. These equally spaced frequency bands allows for better representation of audio.

In some embodiments, a sensed EMF signal is filtered by converting the sensed EMF data into an audio signal or a signal having the morphology of an audio signal wave, and then generating MFCCs.

MFCCs help in identifying the components of the audio signal that are able to differentiate between important content and background noise.

In general, steps for filtering an audio signal derived from sensed EMF data comprise: In a first step, the audio signal is framed into short frames. In a second step, the periodogram estimate of the power spectrum for each frame is calculated. In a third step, a mel filterbank is applied to the power spectrum and sums the energy in each filter. In a fourth step, the logarithm of all the filterbank energies is determined and the DCT of the log filterbank energies is calculated. In a fifth step, only the first 20 DCT coefficients are kept, and the rest are discarded.

Once filtered, the filtered data is transmitted to a machine learning algorithm for analysis. The algorithm described herein is capable of classifying and characterizing the physiological health of human body tissues. The algorithm is designed to analyze input data and determine the presence and location of diseased tissue in the organ(s) recorded by aforementioned sensors.

### Devices and Systems

In some embodiments EMF data is sensed using a device or system. In some embodiments, a device or system comprises one or more EMF sensors. In some of these embodiments, the device or system is configured to include a machine learning software module as described herein. In some of these embodiments, the device or system is configured to transmit a sensed EMF to a machine learning software module not included as part of the device or system. EMF data that is sensed using an electromagnetic sensor comprises electromagnetic data associated with a passage of a current through a cell, tissue, and/or organ of an individual, such as, for example, the heart of the individual. Generally, described herein are devices and systems that comprise digital processing devices.

In some embodiments of devices and systems described herein, a device and/or a system comprises a digital processing device configured to run a software application as described herein. In further embodiments, a digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected to a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, handheld computers, and tablet computers.

In some embodiments, the digital processing device includes an operating system configured to perform executable instructions. The operating system is, for example, software, including programs and data, which manages the device's hardware and provides services for execution of applications. Non-limiting examples of suitable operating systems include FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing.

In some embodiments, a digital processing device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes, optical disk drives, and cloud computing based storage. In further embodiments, the storage and/or memory device is a combination of devices such as those disclosed herein.

In some embodiments, the digital processing device includes a display to send visual information to a subject. In some embodiments, the digital processing device includes an input device to receive information from a subject. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In still further embodiments, the input device is a combination of devices such as those disclosed herein.

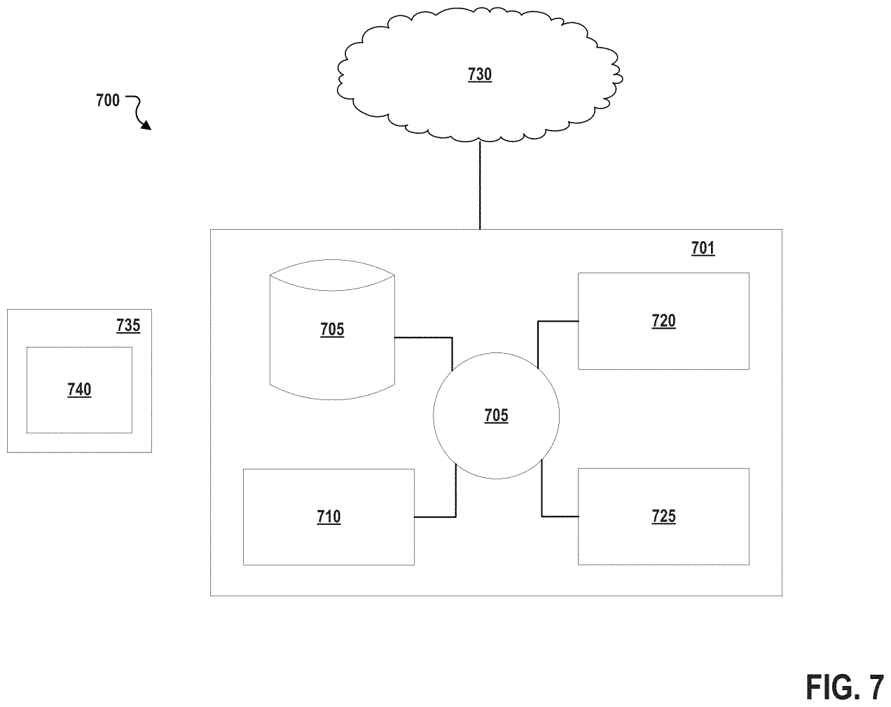

The present disclosure provides computer control systems that are programmed to implement methods of the disclosure. FIG. 7 depicts a computer system 701 that is programmed or otherwise configured to direct operation of a device or system, including movement of a base unit, movement of a shield, movement of a mobile cart, movement of a sensor array, acquisition of a measurement, comparison of a measurement to a reference measurement, or any combination thereof. The computer system 701 can regulate various aspects of (a) movement of one or more device or system components, (b) operation of one or more sensors, (c) adjustment of one or more parameters of a sensor, (d) computationally evaluation of one or more measurements of a device or system, (e) display of various parameters including input parameters, results of a measurement, or any combination of any of these. The computer system 701 can be an electronic device of a user or a computer system that is remotely located with respect to the electronic device. The electronic device can be a mobile electronic device.

The computer system 701 includes a central processing unit (CPU, also “processor” and “computer processor” herein) 705, which can be a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communication bus (solid lines), such as a motherboard. The storage unit 715 can be a data storage unit (or data repository) for storing data. The computer system 701 can be operatively coupled to a computer network (“network”) 730, such as network 110 of FIG. 1, with the aid of the communication interface 720. The network 730 can be the Internet, an internet and/or extranet, or an intranet and/or extranet that is in communication with the Internet. The network 730 in some cases is a telecommunication and/or data network. The network 730 can include one or more computer servers, which can enable distributed computing, such as cloud computing. The network 730, in some cases with the aid of the computer system 701, can implement a peer-to-peer network, which may enable devices coupled to the computer system 701 to behave as a client or a server.

The CPU 705 can execute a sequence of machine-readable instructions, which can be embodied in a program or software. The instructions may be stored in a memory location, such as the memory 710. The instructions can be directed to the CPU 705, which can subsequently program or otherwise configure the CPU 705 to implement methods of the present disclosure. Examples of operations performed by the CPU 705 can include fetch, decode, execute, and writeback.

The CPU 705 can be part of a circuit, such as an integrated circuit. One or more other components of the system 701 can be included in the circuit. In some cases, the circuit is an application specific integrated circuit (ASIC).

The storage unit 715 can store files, such as drivers, libraries and saved programs. The storage unit 715 can store user data, e.g., user preferences and user programs. The computer system 701 in some cases can include one or more additional data storage units that are external to the computer system 701, such as located on a remote server that is in communication with the computer system 701 through an intranet or the Internet.

The computer system 701 can communicate with one or more remote computer systems through the network 730. For instance, the computer system 701 can communicate with a remote computer system of a user (e.g., a second computer system, a server, a smart phone, an ipad, or any combination thereof). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 701 via the network 730.

Methods as described herein can be implemented by way of machine (e.g., computer processor) executable code stored on an electronic storage location of the computer system 701, such as, for example, on the memory 710 or electronic storage unit 715. The machine executable or machine readable code can be provided in the form of software. During use, the code can be executed by the processor 705. In some cases, the code can be retrieved from the storage unit 715 and stored on the memory 710 for ready access by the processor 705. In some situations, the electronic storage unit 715 can be precluded, and machine-executable instructions are stored on memory 710.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 701 can include or be in communication with an electronic display 735 that may comprises a user interface (UI) 740 for providing, for example, a graphical representation of one or more signals measured, one or more reference signals, one or more parameters that may be input or adjusted by a user or by a controller, or any combination thereof. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 705. The algorithm can, for example, comparing a signal to a reference signal.

### Exemplary Applications

The systems, methods, devices, and software described herein are used in a number of different applications including in research and healthcare settings, wherein the systems, methods, devices, and software are used to evaluate a status of an individual and in some cases provide a diagnosis for a condition that the individual has. A condition may comprise both an abnormality (including a pre-disease condition) as well as a disease state. Exemplary types of disease evaluated by the systems, methods, devices, and software described herein include cardiac disease, neurologic disease, and gastrointestinal disease.

In some embodiments, devices, systems, software, and methods described herein provide a suggestion for a next diagnostic step to carry out with the individual following sensing and analyzing the EMF of the individual, such as, for example, an additional diagnostic test or modality that will assist in obtaining a diagnosis. Non-limiting examples of diagnostic modalities suggested include imaging, blood testing, and conduction monitoring (e.g., ECG and EEG).

In some embodiments, devices, systems, software, and methods described herein provide a suggestion for a treatment to be provided to an individual following sensing and analyzing the EMF of the individual.

(a) Cardiac Disease

In some embodiments, the systems, methods, devices, and software described herein are used to evaluate an individual for cardiac disease. Non-limiting examples of cardiac disease evaluated by the systems, methods, devices, and software described herein include CAD, arrhythmia, and congestive heart failure.

In some embodiments, the systems, methods, devices, and software described herein are used to evaluate an individual for CAD. In these embodiments, an EMF associated with a heart of an individual is sensed and based on the sensed EMF of the individual, a status of the individual is determined with respect to CAD. In some of these embodiments, a determination is made as to whether coronary disease is present in the individual. In some of these embodiments, a determination is made as to a degree of severity of a CAD that is present. A degree of severity determined, in some embodiments, comprises “severe,” “moderate,” or “mild,” A degree of severity, in some embodiments, comprises a degree of an obstruction of one or more coronary vessels. For example, in some embodiments, an individual may be determined to have >90% obstruction of their Left Anterior Descending (LAD) artery, >80% obstruction of their LAD, >70% obstruction of their LAD, >60% obstruction of their LAD, or >50% obstruction of their LAD. In some embodiments, the systems, methods, devices, and software described herein determine a presence of a pre-CAD state or that a risk of developing coronary artery exists in the individual. For example, in some embodiments, it is determined that an individual has a >90% risk of developing moderate to severe CAD, a >80% risk of developing moderate to severe CAD, a >70% risk of developing moderate to severe CAD, a >60% risk of developing moderate to severe CAD.

In some embodiments, the systems, methods, devices, and software described herein are used in an acute care setting to evaluate individuals with chest pain. For example, in some embodiments, individuals with left sided chest pain of unknown origin are ruled out of having CAD. For example, in some embodiments, individuals with left sided chest pain of unknown origin are ruled in for having CAD. In some embodiments, an individual with a normal ECG and/or at last one normal troponin level is assessed by the systems, devices, methods, and software described herein and determined to either have CAD, not have CAD, have a high likelihood of having CAD, or have a high likelihood of not having CAD.

More specifically, a system as described herein includes at least one EMF sensor (or a plurality of EMF sensors, or a plurality of EMF sensors arranged in an array) that are positioned in proximity to the heart of an individual. In some embodiments the system further comprises shielding to shield the at least one EMF sensor from ambient EMF readings. Once the at least one sensor senses an EMF, the sensed EMF is analyzed by the software described herein including a machine learning algorithm and a determination is made with respect to the status of the heart of the individual. In some embodiments, the analysis process comprises the generation, by the software described herein, of a visual representation of the EMF that is then analyzed. In some embodiments, a sensed EMF that shows a regular pattern without magnetic dipole dispersion, represents a normal finding, an absence of a presence of CAD in the individual, or a low likelihood of a presence of CAD in the individual. In some embodiments, a sensed EMF that shows an irregular pattern of magnetic pole dispersion represents an abnormal finding, a presence of CAD in the individual, or a high likelihood of a presence of CAD in the individual. In some embodiments, a shift in dipole angulation or significant disorganization in the magnetic field map (e.g., a triple pole) indicates a greater degree of vessel stenosis (i.e., greater degree of CAD).

In some embodiments, a suggestion for a treatment is provided. Non-limiting examples of treatments suggested for CAD include conservative treatment (e.g., improve diet and/or exercise), cholesterol lowering treatment, vasodilating medications, rhythm modulating medications, intravascular interventions including stenting, and bypass surgery.

(b) Neurological Disease

In alternative embodiments, the systems, methods, devices, and software described herein are used to evaluate an individual for neurological disease including abnormalities resulting from traumatic injury and stroke. Non-limiting examples of neurological disorders evaluated by the systems, methods, devices, and software described herein include epilepsy, stroke, traumatic brain injury, traumatic spine injury, encephalitis, meningitis, tumor, Alzheimer's disease, Parkinson's disease, ataxia, and psychiatric disorders including schizophrenia, depression, and bipolar disease.

(c) Gastrointestinal Disease

In alternative embodiments, the systems, methods, devices, and software described herein are used to evaluate an individual for gastrointestinal disease including any disease or disorder of any component of the gastrointestinal system including the gastrointestinal tract, the liver (including biliary system), and the pancreas. Non-limiting examples of gastrointestinal disorders evaluated by the systems, methods, devices, and software described herein include gastrointestinal cancers (including tumors of the gastrointestinal tract, liver, and pancreas), Crohn's disease, ulcerative colitis, irritable bowel disease, dismotility disorders, gall stones, colitis, cholangitis, liver failure, pancreatitis, and infections of the gastrointestinal system.

It should be understood, that any device, system, and/or software described herein is configured for use in or is captured by one or more steps of a method.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A healthcare platform comprising:
(a) an electromagnetic field sensing system configured to sense an electromagnetic field data associated with an individual, wherein the electromagnetic field sensing system comprises sensors configured to non-invasively sense electromagnetic fields generated by a tissue, an organ, or a body part of the individual;
(b) a healthcare provider portal configured to be used by a healthcare provider of the individual;
(c) a patient portal configured to be used by the individual; and
(d) a server configured to operatively communicate with the healthcare provider portal and the patient portal, the server encoded with software modules comprising:
(i) a data ingestion module configured to receive the sensed electromagnetic field data;
(ii) a service module configured to provide at least one healthcare service that is accessed through the healthcare provider portal and the patient portal, the at least one healthcare service related to the sensed electromagnetic field data;
(iii) an interface module configured to provide the healthcare provider portal and the patient portal with access to the at least one healthcare service, the interface module comprising an application programming interface;
(iv) a machine learning module configured to apply a trained machine learning algorithm to the sensed electromagnetic field data, thereby generating an analysis result; and
(v) a data analysis module configured to identify a presence or absence of an abnormality of the tissue, organ, or body part of the individual based on the analysis result.

2. The platform of claim 1, wherein the electromagnetic field sensing system comprises an array of sensors comprising optically pumped magnetometer sensors, magnetic induction sensors, magneto-resistive sensors, superconducting quantum interference device (SQUID) sensors, or a combination thereof.

3. The platform of claim 1, wherein the electromagnetic field sensing system comprises an ambient electromagnetic shield.

4. The platform of claim 3, wherein the ambient electromagnetic shield comprises a bore through which a body of the individual is passed.

5. (canceled)

6. The platform of claim 1, wherein the software modules further comprise a graphic module configured to generate a graphic representation of the sensed electromagnetic field data, and wherein the at least one healthcare service comprises a graphic representation of the sensed electromagnetic field data.

7. (canceled)

8. The platform of claim 1, wherein the at least one healthcare service comprises an interactive electronic medical record or an interactive medical image.

9. (canceled)

10. The platform of claim 1, wherein the at least one healthcare service comprises raw sensed electromagnetic field data.

11. The platform of claim 1, wherein the at least one healthcare service comprises a global reader service configured to provide an interpretation of a medical image.

12. The platform of claim 1, wherein the at least one healthcare service comprises an interactive electronic medical record management service.

13. The platform of claim 1, wherein the at least one healthcare service comprises a machine learning module configured to apply a trained machine learning algorithm to the sensed electromagnetic field data, thereby generating an analysis result, and wherein the data analysis module is further configured to determine a diagnosis of the individual based on the analysis result.

14. (canceled)

15. The platform of claim 1, wherein the at least one healthcare service comprises a software module configured to generate an electric current map based on the sensed electromagnetic field data.

16. The platform of claim 1, wherein the healthcare provider portal comprises a communication interface configured to provide at least one of a text, an audio, and a video transmission from the healthcare provider portal to the patient portal.

17. The platform of claim 1, wherein the patient portal comprises a communication interface configured to provide at least one of a text, an audio, and a video transmission from the patient portal to another patient portal.

18. The platform of claim 1, wherein the application programming interface comprises a portal for encoding protocols for a behavior of the interface module.

19. The platform of claim 18, wherein the protocols are configured to cause the software modules to integrate with a customized healthcare provider portal and a customized patient portal.

20. (canceled)

21. The platform of claim 18, wherein the protocols are configured to generate a user authentication system.

22. The platform of claim 1, wherein the tissue, organ, or body part is a heart of the individual.

23-44. (canceled)

45. The platform of claim 1, wherein the machine learning module comprises a multi-layer neural network.

46. The platform of claim 45, wherein the multi-layer neural network comprises a plurality of dilated convolutional neural networks.

47. The platform of claim 1, wherein the software modules further comprise an encoding module configured to encode the sensed electromagnetic field data into a plurality of time-correlated independent components, and wherein the machine learning module is configured to further apply the trained machine learning algorithm to the plurality of time correlated independent components to generate the analysis result.

48. The platform of claim 6, wherein the graphic representation of the sensed electromagnetic field data comprises a magnetocardiogram.

49. The platform of claim 22, wherein the data analysis module is further configured to identify the presence or absence of a cardiac disease of the individual.

50. The platform of claim 49, wherein the cardiac disease comprises coronary artery disease (CAD).
